(19) United States
(12) Patent Application Publication
(10) Pub. No.: US 2015/0202341 A1
(43) Pub. Date: Jul. 23, 2015

(54) PHOTO CATALYTIC AIR PURIFIER

(71) Applicant: Vornado Air, LLC, Andover, KS (US)

(72) Inventors: Glen W. Ediger, North Newton, KS (US); Gary P. Israel, Andover, KS (US)

(21) Appl. No.: 14/418,443

(22) PCT Filed: Jul. 30, 2013
(86) PCT No.: PCT/US13/52666

§ 371 Date: Jan. 30, 2015

Related U.S. Application Data
(60) Provisional application No. 61/677,073, filed on Jul. 30, 2012.

(51) Int. Cl. A61L 9/20; F24F 3/16

(52) U.S. Cl. CPC A61L 9/205; F24F 3/1603; F24F 2003/1628; F24F 2003/1667; F24F 2003/1689

(57) ABSTRACT

An air purification system includes a housing having an inlet and a panel. The panel at least partially defines a volume for receiving air from the inlet. The system also includes an internal support structure coupled to the housing and configured to support a plurality of components of the air purifier. The system further includes a blower configured to drive air through the air purifier components. The system also includes a first drawer including a mesh screen having a photocatalytic oxidation catalyst disposed on its surface. The first mesh screen is placed within the flow of air. The system further includes a second drawer including an ultraviolet light bulb in line-of-site position relative to the mesh screen. The system also includes a light shield configured to eliminate line-of-site access to the ultraviolet light bulb from an outlet of the air purification system.

### TECHNICAL FIELD

The present disclosure relates to air purifiers. In particular, the present disclosure relates to air purifiers utilizing photocatalytic oxidation to remove various impurities.

### BACKGROUND

Indoor air quality can be substantially impaired by the presence of various contaminants. In fact, the result of recirculation of indoor air through a partially closed system can result in the presence of contaminants at level significantly higher than in the outdoor air. As a result, recirculated air is typically passed through a filter capable of removing at least some of these contaminants. However, filters alone are not effective for the removal of all common contaminants.

For example, volatile organic compounds (VOCs) are a class of compounds that are typically in gas form within the air. As these are recirculated, their concentration can increase because conventional filters may be ineffective at entraining them. VOCs, in sufficient concentration, may cause headaches, bad odors, and other undesirable results. VOCs may be introduced into an indoor environment by a variety of sources. These include fabrics, solvents, adhesives, cleaning agents, and vehicle exhaust.

The result can be poor air quality that is not sufficiently addressed by conventional air filters. One known method of removing VOCs and other contaminants from the air is photocatalytic oxidation. Such systems utilize a photo catalyst, typically disposed on a substrate, and a source of ultra violet light. The catalyst, which may comprise metal oxides and/or hydroxides, retains the VOC molecules by one or more of typical catalytic actions. While held in place by the catalyst, the VOC molecules are bombarded with UV light. The energy from the light, combined with oxygen in the air, results in the oxidation of the VOCs into carbon dioxide and water. The carbon dioxide and water are have less affinity for the catalyst than the VOC molecules, so they are quickly entrained in the air stream leaving the air purifier.

Typical systems utilizing photocatalytic oxidation are cumbersome and suitable only for fixed, permanent installation in a home or business heating, ventilation, and air conditioning (HVAC) system. Such systems are also often very specialized and require a skilled technician to perform routine maintenance. Accordingly, there is a need for an air purification system utilizing photocatalytic oxidation that can be used in a room within a home or business. There is yet another need for an air purification system utilizing photocatalytic oxidation that is modular in nature and can allow for routine maintenance to be carried out by the consumer.

### SUMMARY

An air purification system includes a housing having an inlet and a panel. The panel at least partially defines a volume for receiving air from the inlet. The system also includes an internal support structure coupled to the housing and configured to support a plurality of components of the air purifier. The system further includes a blower configured to drive air through the air purifier components. The system also includes a first drawer including a mesh screen having a photocatalytic oxidation catalyst disposed on its surface. The first mesh screen is placed within the flow of air. The system further includes a second drawer including an ultraviolet light bulb in line-of-site position relative to the mesh screen. The system also includes a light shield configured to eliminate line-of-site access to the ultraviolet light bulb from an outlet of the air purification system.

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

### DRAWINGS

The Detailed Description is described with reference to the accompanying figures. The use of the same reference numbers in different instances in the description and the figures may indicate similar or identical items.

FIG. 1 is a perspective view illustrating an air purification system in accordance with an example embodiment of the present disclosure.

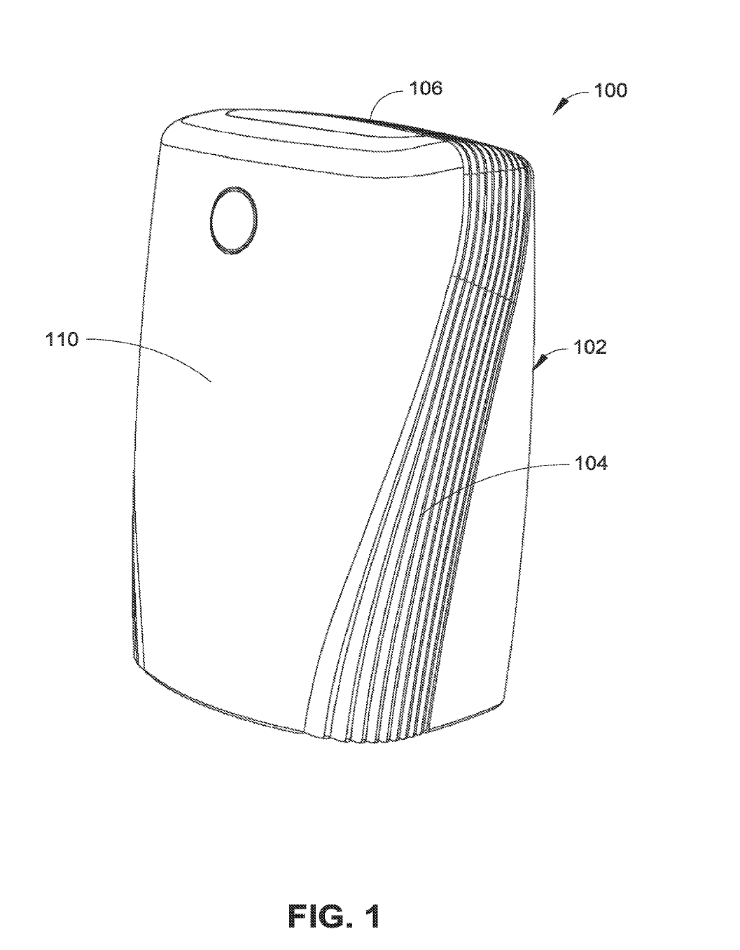

FIG. 2 is a side cutaway view of the air purification system illustrated in FIG. 1.

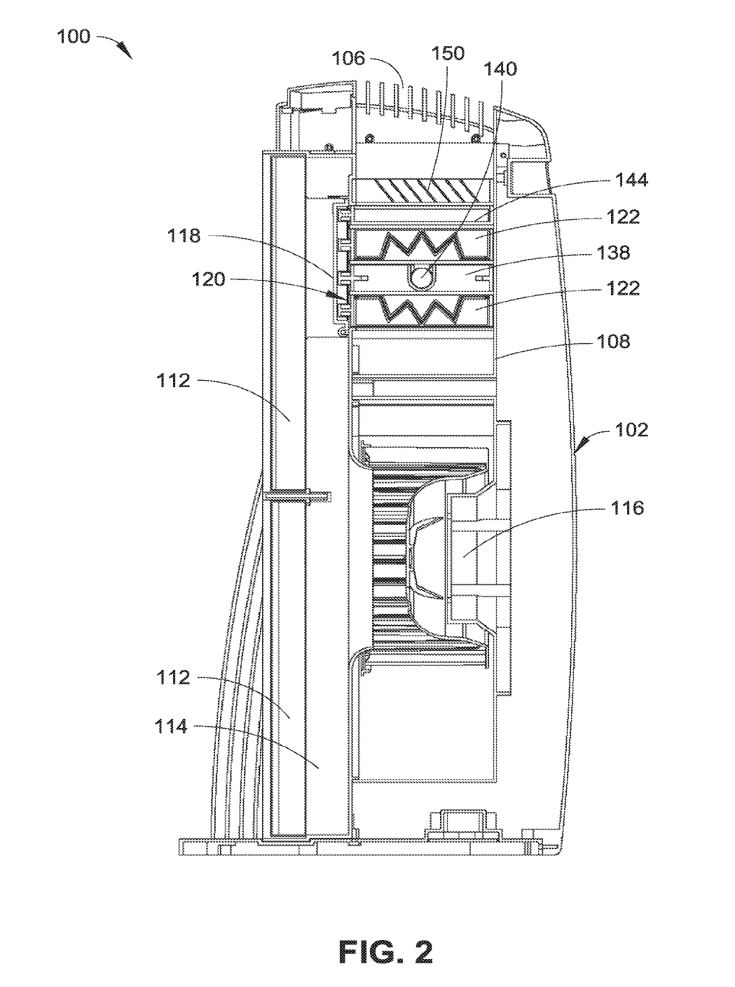

FIG. 3 is a partial perspective view of the air purification system illustrated in FIG. 1.

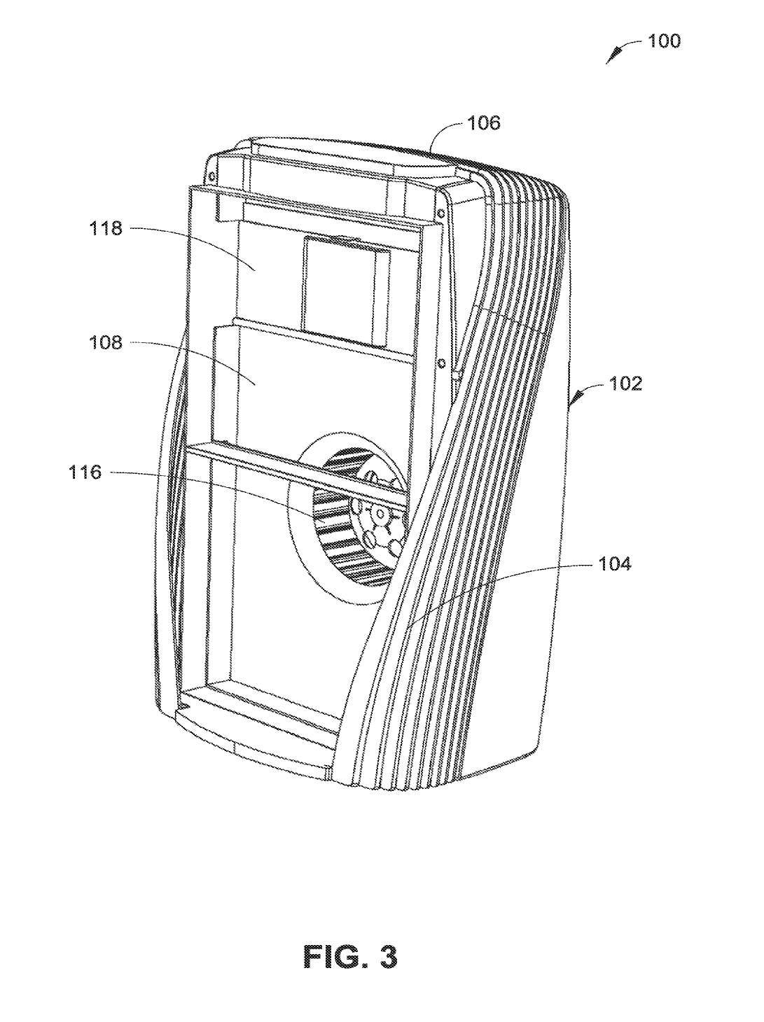

FIG. 4 is a partially exploded perspective view of the air purification system illustrated in FIG. 1.

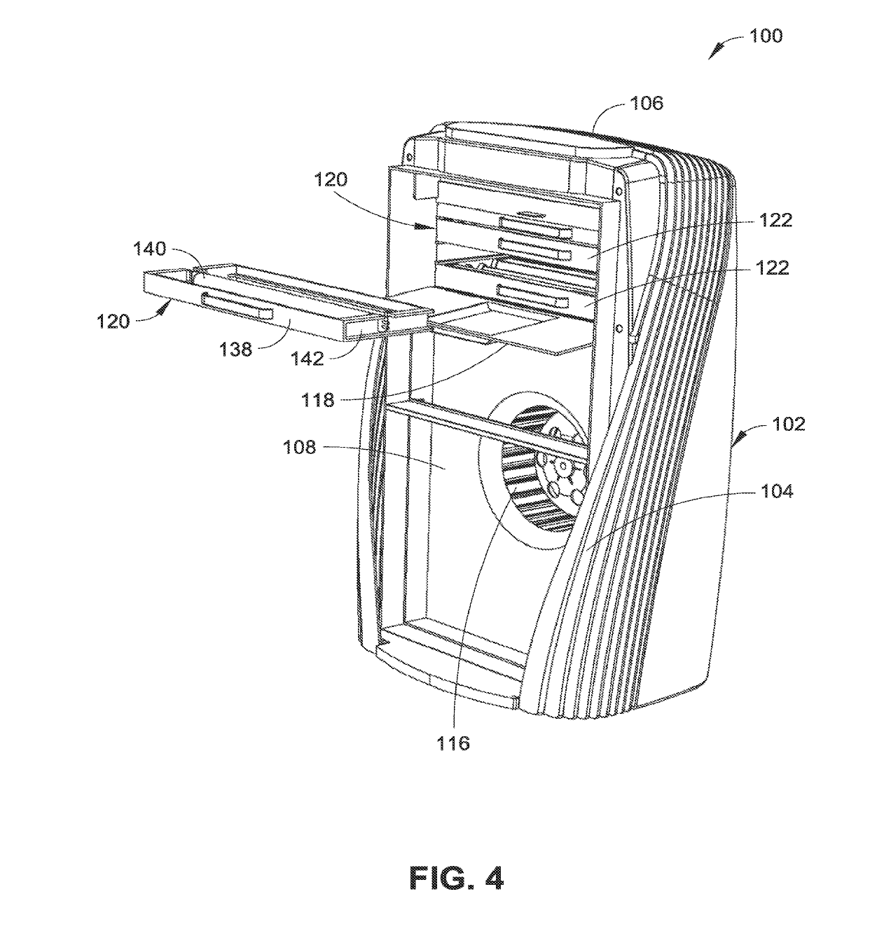

FIG. 5 is another partially exploded perspective view of the air purification system illustrated in FIG. 1.

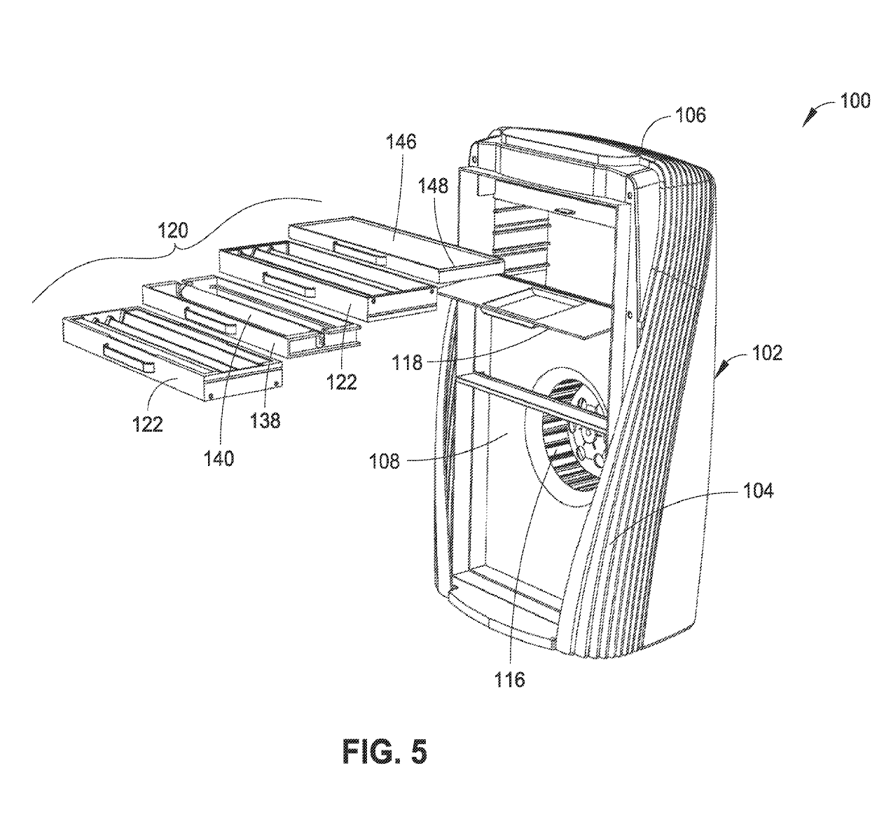

FIG. 6 is a perspective view illustrating a drawer for retaining a catalytic mesh within an air purification system, such as the air purification system illustrated in FIG. 1, in accordance with an example embodiment of the present disclosure.

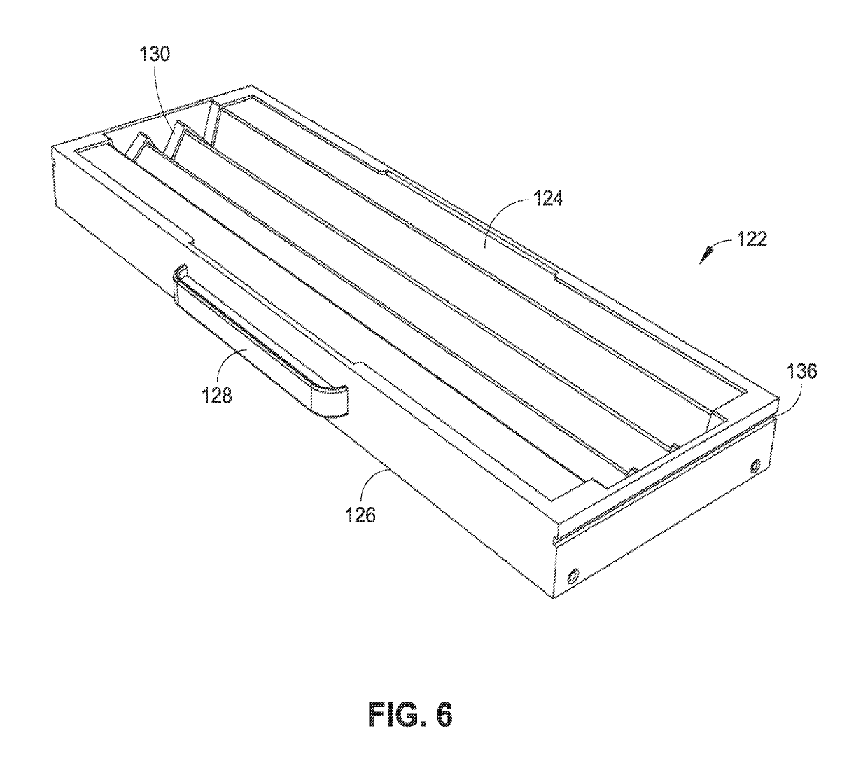

FIG. 7 is a partially exploded perspective view of the drawer illustrated in FIG. 6.

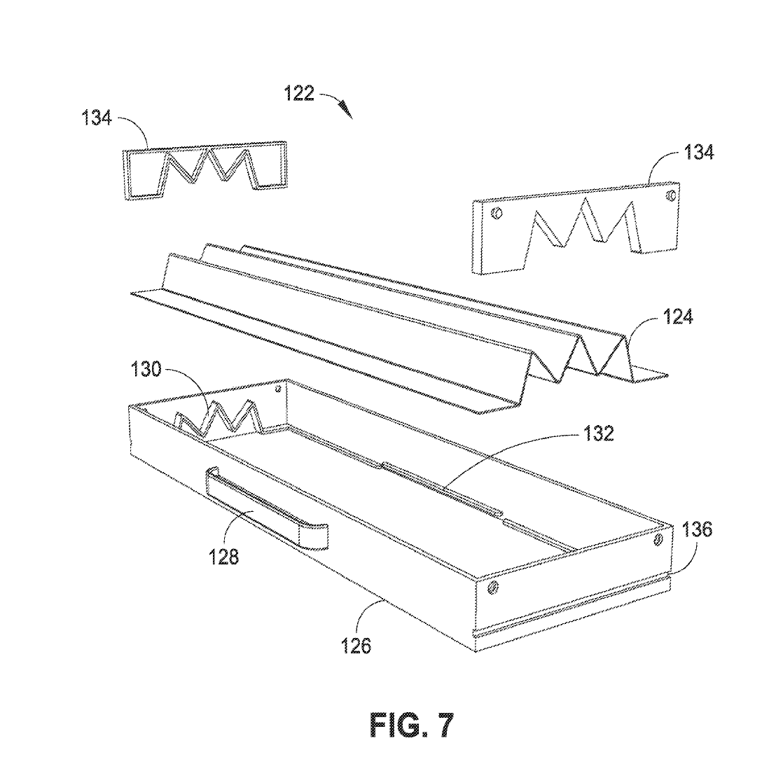

FIG. 8 is a partial cutaway perspective view of the air purification system illustrated in FIG. 1.

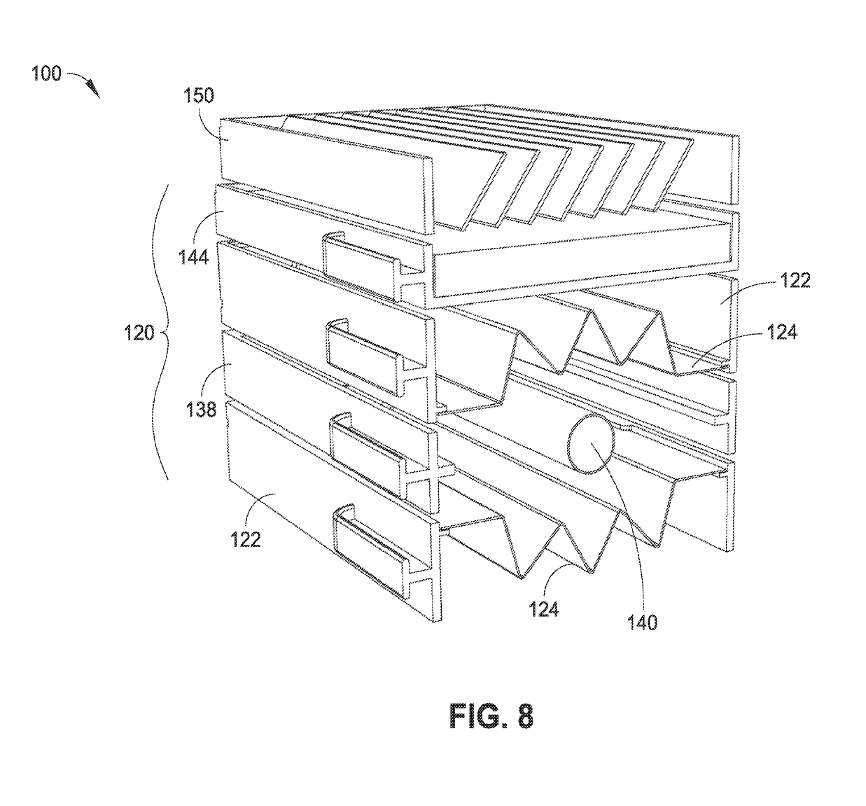

### DETAILED DESCRIPTION

A stand alone, consumer air purifying system 100 includes a housing 102 having side air inlets 104 and a top air outlet 106 and an internal support structure 108 for holding the various components of the system 100. A set of controls to regulate the flow of air may also be provided on the housing 102. The housing 102 includes a selectively removable front panel 110 to provide access to the interior of the system 100. Air inlets 104 may be positioned on the sides of the system 100, or at any other usable position, to allow outside air into the purifier system 100. As shown, the inlets 104 allow air to be drawn into a region interior to the front panel 110. The air may then pass through one or more pre-filters.

The pre-filters may be any conventional filter for the removal of particulate matter from the inlet air. In particular, the pre-filters may comprise a washable, mesh filter that may be cleaned and/or replaced as needed. The pre-filters may aid in prolonging the useful life of other, more costly components that may otherwise need to be replaced due to heavy particulate loads. Placing the pre-filters upstream of the blower can also avoid damage to the blower by particulates.

After passing through the pre-filter(s), the inlet air may be drawn through one or more high efficiency particulate air (HEPA) filters 112. The interior of the purifier may be configured to allow two standard sized HEPA filters 112 to be placed in the flow of air. The HEPA filters 112 are typical construction and are supported by an internal plastic rectangular structure. A gasket is included with a HEPA filter 112 that seals the gap between the HEPA filter 112 and support structure 108. In some embodiments, the system 100 includes two pre-filters and two HEPA filters 112 with a separator support between the two HEPA filters 112.

Behind the HEPA filters 112 is a plenum area 114 which allows space for the air behind the HEPA filters 112 to be drawn into the blower area. A centrifugal blower, such as a forward curved centrifugal blower 116 is of typical construction and is turned by an electric motor which is supported by an internal support structure 108. The blower 116 turns inside a typical volute design that provides air-flow performance. The air may then be directed in an upward direction through rectangular shaped ductwork. In this rectangular ductwork area are located filtration components along with an odor eliminating filter and a light deflector.

The filtration components are configured to allow component replacement and to provide a geometry that provides increased functionality of the components. To allow access to the components, first the front panel 110 is removed. When the panel 110 is removed, an internal switch may cut all power to the internal components to comply with safety requirements for accessibility to moving parts as well as electrical components and exposure to the UV bulb.

With the front panel 110 removed, the pre-filters and HEPA filters 112 can then be removed. In turn, an internal door 118 may be accessed. This door 118 may be hinged at the bottom, and have a snap fit at the top. The door 118 is generally configured to cover all the drawers 120 that hold the filtration components. This door 118, when closed, will also assure that all drawers 120 are completely pushed in. This door 118 also provides complete line-of-site containment of the UV bulb.

With the access door 118 open any of the drawers 120 may be removed for replacement. There is a limited life cycle for each component. For example the light may last about 1 year, the odor filter may need to be replaced about every six months or one year, and the titanium dioxide (TiO₂) coated screen mesh may need to be replaced about every 5 years.

One or more mesh filter drawers 122 may be provided. A drawer assembly is designed to support a TiO₂ coated mesh screen 124 in the proper orientation relative to the UV light. By providing two mesh filter drawers 122, the mesh screens 124 may be positioned both below and above the light for increased surface area exposed to the light. In embodiments of the disclosure, the mesh screen 124 is also configured with an increased exposed area to the bulb by incorporating a pleated zigzag design located in a slight arch shape so as to expose all mesh to the direct light and increase the exposure of the light to the available surface area. This mesh filter drawer 122 may be injection molded in construction, comprising a frame 126, with molded in drawer handle 128, a zigzag support 130 at each end, and a mid-support 132 on each long side that contains the mesh screen 124 in both directions. There is also an end cap 134, which is also injection molded with the mating zigzag that snaps into location at each end securing the mesh screen 124 into proper location. The mesh filter drawer 122 may also be provided with orientation grooves 136 on the sides to assure discrete orientation into the proper opening and in the proper direction. This assures that the mesh filter drawer 122 below the bulb faces up and the mesh filter drawer 122 above the bulb faces down. In some embodiments, the mesh filter drawer 122 and end caps 134 are molded of UV resistant plastic materials. In some configurations only one mesh filter drawer 122 may also be used.

A light drawer 138 may also be provided for mounting a light bulb (e.g., a UV bulb 140). The light drawer 138 may be configured to hold the UV bulb 140 in position in the system 100 and in location relative to the one or more mesh screens 124. In some embodiments, to reduce anxiety of handling the UV bulb 140 and make the replacement process easier, the light drawer 138 can be removed, the UV bulb 140 removed and replaced with a new UV bulb 140, and then the light drawer 138 with new UV bulb 140 may be replaced into the system 100. When the light drawer 138 is placed into the system 100, it may automatically slide into an electrical connection needed to energize the bulb in use. The light drawer 138 may be injection molded of UV resistant plastic materials, and the light drawer 138 is provided with orientation grooves 142 on the sides to assure discrete orientation into the proper opening and in the proper direction.

In some embodiments, a nano-filter drawer 144 may be provided. This nano-filter drawer 144 is designed to support a replaceable nano odor filter 146. However filters other than nano technology filters may also be used. This drawer 144 holds the filter in proper location downstream from the filtration components. The drawer 144 may be easily removed to allow replacement of the filter 146. The drawer 144 may be injection molded of UV resistant plastic materials, and drawer 144 is provided with orientation grooves 148 on the sides to assure discrete orientation into the proper opening and in the proper direction.

To reduce the risk of exposure to UV light, a light shield 150 may also be provided. In some embodiments, the light shield 150 is not removable. The light shield 150 is included downstream from the filtration components and, in combination with the outlet grill, blocks line-of-site visible access to the UV bulb 140. This component is injection molded of UV resistant plastic and black in color to minimize reflection of the UV light.

The use of drawer assemblies allows for the simplified and selective replacement of components in the air purification system 100. This allows the user to make replacements of individual parts as needed. Accordingly, parts can be used to their potential for their useful life, rather than being replaced prematurely when other parts need to be replaced as part of a larger assembly.

Although the subject matter has been described in language specific to structural features and/or process operations, it is to be understood that the subject matter defined in the appended claims is not necessarily limited to the specific features or acts described above. Rather, the specific features and acts described above are disclosed as example forms of implementing the claims.

What is claimed is:

1. An air purification system comprising:
a housing having an inlet and a panel, the panel at least partially defining a volume for receiving air from the inlet;
an internal support structure coupled to the housing and configured to support a plurality of components of the air purifier;
a blower configured to drive air through the air purifier components;
a first drawer including a mesh screen having a photocatalytic oxidation catalyst disposed on its surface, the first mesh screen placed within the flow of air;
a second drawer including an ultraviolet light bulb in line-of-site position relative to the mesh screen; and
a light shield configured to eliminate line-of-site access to the ultraviolet light bulb from an outlet of the air purification system.

2. The air purification system of claim 1, further comprising a third drawer including a second mesh screen having a photocatalytic oxidation catalyst disposed on the surface of the second mesh screen, placed within the flow of air and in line of site position relative to the ultraviolet bulb.

3. The air purification system of claim 2, wherein one of the first mesh screen and the second mesh screen is disposed upstream of the ultraviolet light bulb and the other of the first mesh screen and the second mesh screen is disposed downstream of the ultraviolet light bulb.

4. The air purification system of claim 2, further comprising a fourth drawer having an odor eliminating filter positioned downstream of the ultraviolet light bulb.
